(19) United States
(12) Patent Application Publication
(10) Pub. No.: US 2023/0310380 A1
(43) Pub. Date: Oct. 5, 2023

(54) METHODS, DEVICES, AND SYSTEMS FOR TREATING LENS PROTEIN AGGREGATION DISEASES

(71) Applicant: Alan Neil Glazier, Rockville, MD (US)

(72) Inventor: Alan Neil Glazier, Rockville, MD (US)

(21) Appl. No.: 17/713,691

(22) Filed: Apr. 5, 2022

(51) Int. Cl. A61K 31/4184; A61K 9/00; A61K 45/06

(52) U.S. Cl. CPC A61K 31/4184; A61K 9/0048; A61K 45/06

(57) ABSTRACT

Disclosed herein are methods, devices, and systems for treating lens protein aggregation diseases by reducing the formation of proteins responsible for crowding, compacting, and/or causing increased internal lens pressure. Specifically disclosed herein are ophthalmologic compositions comprising one or more agents that regulate water, sodium, and/or calcium ion transport and/or storage through lens fiber cell channels, and/or reduce the formation and/or production of proteins responsible for crowding, compacting, and/or causing increased internal lens pressure.

### FIELD

The application relates generally to methods, devices, and systems for treating lens protein aggregation diseases. In particular, the application relates to novel methods and compositions for reducing the formation of proteins responsible for crowding, compacting, and/or causing increased internal lens pressure.

### BACKGROUND

Mammalian lens protein aggregation diseases affect the human eye, including presbyopia and cataract. For the average healthy (e.g., non-diabetic, non-smoking) individual, presbyopia can manifest clinically in the early 40's as difficulty seeing objects at close range. However, the processes that lead to presbyopia often begins decades before any clinical symptoms are evident.

One of the aforementioned processes leads to a dramatic increase in lens stiffness as an individual ages. For instance, the nucleus, which is a part of the eye lens, becomes approximately 500- to 1000-fold stiffer over the average person's lifetime. Generally, the onset of symptoms associated with presbyopia and other lens aggregation diseases are attributed to the loss of natural enzymatic and antioxidant protection in the eye against, for instance, ultraviolet A (UVA) and ultraviolet B (UV B) radiation, with a concurrent increase in the production of photochemically active chromophores (oxidants).

Accordingly, the key cause of presbyopia and, ultimately, cataractogenesis, is believed to be multifactorial, influenced by a combination of endogenous and exogenous oxidation. Endogenous oxidation occurs via internal mechanisms (e.g., intraocular photochemical generation of free radicals and other oxidants), while exogenous oxidation may be due to exposure to environmental causes (e.g., an increased exposure over an individual's lifespan to short wavelength and ultraviolet (UV) radiation, chemical ingestion (such as smoking), diabetes, and the like).

However, the theory for oxidation as the root cause of presbyopia and other mammalian lens aggregation diseases cannot alone account for changes that result in protein aggregation (e.g., an increase in lens pressure, a decrease in lens flexibility). Such changes may play a more significant role in lens aggregation diseases than currently acknowledged.

Given the foregoing, there exists a significant need for novel technology that manages (e.g., maintains and/or reduces) internal lens pressure, thus reducing onset and/or treating lens protein aggregation diseases, such as, for instance, presbyopia and cataracts.

### SUMMARY

It is to be understood that both the following summary and the detailed description are exemplary and explanatory and are intended to provide further explanation of the invention as claimed. Neither the summary nor the description that follows is intended to define or limit the scope of the invention to the particular features mentioned in the summary or in the description.

In general, the present disclosure is directed towards methods and compositions for reducing internal lens pressure. In particular, the disclosure relates to topical and/or injectable treatments (e.g., eyedrops) for reducing the presence and/or formation of proteins responsible for crowding, compacting, and/or causing increased internal lens pressure.

In one or more of the embodiments described herein, an ophthalmologic composition is disclosed that reduces the effects of, retards, and/or eliminates one or more lens protein aggregation diseases (e.g., presbyopia, cataract). One or more of the compositions disclosed herein may be administered via injection (e.g., retrobulbar injection, injection directly into the eye) in the form of a solution, suspension, emulsion, gel, prodrug, ointment, and/or sustained release vehicle (e.g., punctal plug, contact lens). Further, one or more such compositions may be administered directly into, and/or target, the lens of the eye, and more specifically the epithelium of the lens of the eye.

In at least one embodiment, an ophthalmologic composition is disclosed that regulates water, sodium, and/or calcium ion transport and/or storage through lens fiber cell channels. Without wishing to be bound by theory, the aforementioned results in minimizing and/or eliminating oxidative damage of gap junction channels and/or upregulating gap junction coupling. The composition may therefore comprise one or more selective inhibitors of calcium ion (Ca²⁺) uptake into the endoplasmic reticulum (ER).

The composition may, in at least one embodiment, comprise one or more non-selective cation channel blockers.

The composition may, in at least one embodiment, comprise one or more compounds that decrease activation and/or inhibit lens tyrosine kinases.

The composition may, in at least one embodiment, comprise one or more agents that block calmodulin (CaM) binding to RyR2 (e.g., calmodulin antagonists).

The composition may, in at least one embodiment, comprise one or more agonists of CaM-dependent phosphodiesterase activity.

The composition may, in at least one embodiment, comprise one or more agents that inhibit expression of calmodulin.

The composition may, in at least one embodiment, comprise one or more agents that suppress Thapsigargin.

The composition may, in at least one embodiment, comprise one or more agents that enhance the activity of, and/or upregulate, adenylate cyclase (AC) activity.

The composition may, in at least one embodiment, comprise one or more agents that induce anti-calpain activity.

The composition may, in at least one embodiment, comprise one or more Transglutaminase (TGase) inhibitors.

The composition may, in at least one embodiment, comprise one or more antioxidants and/or inhibitors of reactive oxygen species-generating enzymes (e.g., nitric oxide synthetase inhibitors).

The composition may, in at least one embodiment, comprise one or more inhibitors of Myosin Light Chain (MLC) Kinase.

The composition may, in at least one embodiment, comprise one or more Inositol 1,4,5-Trisphosphate (InsP₃) receptor inhibitors.

The composition may, in at least one embodiment, comprise one or more Inositol 1,4,5-trisphosphate 3-kinase (IP3K) inhibitors and/or one or more anti-IP3K-specific antibodies.

The composition may, in at least one embodiment, comprise one or more agents that reduce gap junction uncoupling and/or increase gap junction coupling.

The composition may, in at least one embodiment, comprise one or more agents that inhibit and/or downregulate protein kinase Cγ (pkCγ).

The composition may, in at least one embodiment, comprise one or more ligands that activate G-protein signaling molecules.

The composition may, in at least one embodiment, comprise one or more agents that activate Protein Kinase A (PKA) (e.g., cAMP agonists, PKA activators).

The composition may, in at least one embodiment, comprise one or more agents that inhibit and/or downregulate cAMP-dependent PDE.

The composition may, in at least one embodiment, comprise one or more agents that activate, stimulate, and/or upregulate Calpastatin.

The composition may, in at least one embodiment, comprise one or more agents that control the activity of Calcium-dependent proteases (e.g., m-calpain, Lp82).

The composition may, in at least one embodiment, comprise one or more agents that inhibit the action of one or more proteases.

The composition may, in at least one embodiment, comprise one or more agents that decrease phosphorylation of α,β-crystallin and/or human lymphatic endothelial cells (HLECs) such as, for example, one or more inhibitors of various tyrosine kinases (e.g., by applying Inhibitors of the tyrosine kinases including MLC Kinase, SRC Kinase, and MAP Kinase (MAPK) p38 kinase).

The composition may, in at least one embodiment, comprise one or more agents that disable ER signaling.

The composition may, in at least one embodiment, comprise one or more agents that reduce or eliminate the breakdown of Acetylcholine in the lens of the eye.

The composition may, in at least one embodiment, comprise one or more Cholinesterase inhibitors.

The composition may, in at least one embodiment, comprise one or more muscarinic agonists.

In at least a further embodiment of the disclosure, methods are disclosed for using one or more of the compositions described herein as a treatment for one or more lens aggregation diseases (e.g., presbyopia, cataract). One or more such methods may comprise placing one or more of the compositions in a solution, suspension, emulsion, gel, prodrug, ointment, and/or sustained release vehicle (e.g., punctal plug, contact lens), and administering the one or more compositions to a patient having one or more lens aggregation diseases (e.g., by administering directly into the eye, the lens of the eye, and/or the epithelium of the lens of the eye).

These and further and other objects and features of the invention are apparent in the disclosure, which includes the above and ongoing written specification, as well as the drawings.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIGS. 1A-1C show one or more ophthalmologic compositions containing agents (FIG. 1A, FIG. 1B, and FIG. 1C) that perform various functions to reduce formation of proteins responsible for crowding, compacting, and/or causing increased lens pressure, according to at least one embodiment of the present disclosure.

FIG. 2 is a diagram of a method for treating a subject having one or more lens aggregation diseases, according to at least one embodiment of the present disclosure.

FIG. 3 is a diagram of a further method for treating a subject having one or more lens aggregation diseases, according to at least one embodiment of the present disclosure.

### DETAILED DESCRIPTION

The present invention is more fully described below with reference to the accompanying figures. The following description is exemplary in that several embodiments are described (e.g., by use of the terms “preferably,” “for example,” or “in one embodiment”); however, such should not be viewed as limiting or as setting forth the only embodiments of the present invention, as the invention encompasses other embodiments not specifically recited in this description, including alternatives, modifications, and equivalents within the spirit and scope of the invention. Further, the use of the terms “invention,” “present invention,” “embodiment,” and similar terms throughout the description are used broadly and not intended to mean that the invention requires, or is limited to, any particular aspect being described or that such description is the only manner in which the invention may be made or used. Additionally, the invention may be described in the context of specific applications; however, the invention may be used in a variety of applications not specifically described.

The embodiment(s) described, and references in the specification to “one embodiment”, “an embodiment”, “an example embodiment”, etc., indicate that the embodiment(s) described may include a particular feature, structure, or characteristic. Such phrases are not necessarily referring to the same embodiment. When a particular feature, structure, or characteristic is described in connection with an embodiment, persons skilled in the art may effect such feature, structure, or characteristic in connection with other embodiments whether or not explicitly described.

In the several figures, like reference numerals may be used for like elements having like functions even in different drawings. The embodiments described, and their detailed construction and elements, are merely provided to assist in a comprehensive understanding of the invention. Thus, it is apparent that the present invention can be carried out in a variety of ways, and does not require any of the specific features described herein. Also, well-known functions or constructions are not described in detail since they would obscure the invention with unnecessary detail. Any signal arrows in the drawings/figures should be considered only as exemplary, and not limiting, unless otherwise specifically noted. Further, the description is not to be taken in a limiting sense, but is made merely for the purpose of illustrating the general principles of the invention, since the scope of the invention is best defined by the appended claims.

It will be understood that, although the terms first, second, etc. may be used herein to describe various elements, these elements should not be limited by these terms. These terms are only used to distinguish one element from another. Purely as a non-limiting example, a first element could be termed a second element, and, similarly, a second element could be termed a first element, without departing from the scope of example embodiments. As used herein, the term “and/or” includes any and all combinations of one or more of the associated listed items. As used herein, “at least one of A, B, and C” indicates A or B or C or any combination thereof. As used herein, the singular forms “a”, “an,” and “the” are intended to include the plural forms as well, unless the context clearly indicates otherwise. It should also be noted that, in some alternative implementations, the functions and/or acts noted may occur out of the order as represented in at least one of the several figures. Purely as a non-limiting example, two figures shown in succession may in fact be executed substantially concurrently or may sometimes be executed in the reverse order, depending upon the functionality and/or acts described or depicted.

As used herein, ranges are used herein in shorthand, so as to avoid having to list and describe each and every value within the range. Any appropriate value within the range can be selected, where appropriate, as the upper value, lower value, or the terminus of the range.

Unless indicated to the contrary, numerical parameters set forth herein are approximations that can vary depending upon the desired properties sought to be obtained. At the very least, and not as an attempt to limit the application of the doctrine of equivalents to the scope of any claims, each numerical parameter should be construed in light of the number of significant digits and ordinary rounding approaches.

The words “comprise”, “comprises”, and “comprising” are to be interpreted inclusively rather than exclusively. Likewise the terms “include”, “including” and “or” should all be construed to be inclusive, unless such a construction is clearly prohibited from the context. The terms “comprising” or “including” are intended to include embodiments encompassed by the terms “consisting essentially of” and “consisting of”. Similarly, the term “consisting essentially of” is intended to include embodiments encompassed by the term “consisting of”. Although having distinct meanings, the terms “comprising”, “having”, “containing” and “consisting of” may be replaced with one another throughout the description of the invention.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

“Typically” or “optionally” means that the subsequently described event or circumstance may or may not occur, and that the description includes instances where said event or circumstance occurs and instances where it does not.

Wherever the phrase “for example,” “such as,” “including” and the like are used herein, the phrase “and without limitation” is understood to follow unless explicitly stated otherwise.

Generally, embodiments of the present disclosure are directed towards novel methods, devices, and systems that maintain or reduce internal lens pressure. In particular, the present disclosure relates to novel methods and compositions for reducing the formation of proteins responsible for crowding, compacting, and/or causing increased internal lens pressure.

Lens Aggregation Diseases

Generally, lens aggregation diseases (e.g., presbyopia) manifest clinically in middle age. However, the processes that lead to presbyopia result in a dramatic increase in lens stiffness, which occurs well before the clinical onset of symptoms.

This onset of symptoms is commonly attributed, at least in part, to the loss of natural enzymatic and antioxidant protection in the eye against ultraviolet radiation (e.g., ultraviolet A (UV-A) and ultraviolet B (UV-B) radiation), with a concurrent increase in the production of photochemically active chromophores or oxidants over a period of time.

One skilled in the art will recognize that, as the lens of the eye absorbs light, chromophores are photoactivated and produce reactive oxygen species (e.g., singlet oxygen and superoxide). These oxidants denature lens proteins. At the same time as the lens accumulates such oxidants and/or oxidized components, there is a decreased efficiency of naturally-occurring mechanisms which repair proteins damaged by oxidation.

As a result, oxidative damage can cause progressive hardening of the lens substance, eventually reaching a level where the lens loses its ability to bend in order to focus, the focusing occurring through a process known in the art, and referred to herein, as “accommodation,” and the gradual stiffening causing the loss of ability to bend, which happens approximately between the ages of 42 and continues to around age 57 (known in the art, and referred to herein, as “presbyopia”). Gradual opacification of the lens results in a decreased amount of available light to the retina, thereby resulting in decreasing vision and/or cataract. This occurs, on average, between the ages of 65 and 75. Cataract, if left untreated, will result in surgically reversible blindness.

The cause of various lens aggregation diseases (e.g., presbyopia and cataractogenesis) is believed to be multi-factorial, and influenced by a combination of endogenous oxidation and exogenous oxidation. “Endogenous oxidation” means oxidation that occurs via internal mechanisms such as, for example, oxidation that occurs intraocular photochemical generation of superoxide and its derivatization to other oxidants such as singlet oxygen, hydroxyl radical, hydrogen peroxide and glycation, loss of the lens' natural ultraviolet (UV) filters, and decreasing amounts of natural lens antioxidants, (e.g., glutathione). “Exogenous oxidation” means oxidation due to external and/or environmental factors, such as, for instance, increased exposure to short wavelength and UV radiation, ingestion of chemicals and pollutants, smoking, diseases such as diabetes, and the like.

It is commonly believed in the art that many types of oxidation, including those mentioned previously herein, denature healthy proteins, leading to a gradual loss of lens elasticity, which results in difficulty focusing at close distances (e.g., as occurs in presbyopia) and/or lens opacification (e.g., as results from cataracts).

However, lens aggregation diseases may not be solely, or mainly, initiated by oxidation; thus, such oxidation may not be the root cause of these diseases. While endogenous and exogenous oxidation are factors, other factors that are not recognized and/or under-recognized in the art include the effects on proteins from rising internal lens pressure.

For example, optical clarity of the lens is generally maintained through the process of normal protein folding and unfolding with respect to lens proteins. Healthy proteins fold, unfold, and refold with the help of so-called “molecular chaperones,” a term known in the art for proteins that assist in healthy folding and/or unfolding. Proteins may misfold and/or unfold due to a variety of factors, such as, for instance, oxidation, declining amounts of chaperones, and other factors. Misfolding may lead to protein aggregation, which, with respect to lens aggregation diseases, gradually hardens and opacifies the lens, thereby causing and/or exacerbating such diseases (e.g., presbyopia, cataracts).

Thus, conventional solutions have failed to adequately consider factors and/or changes other than exogenous and endogenous oxidation that may contribute to the onset and/or development of lens aggregation diseases.

Lens Pressure

Purely as a non-limiting example, changes in internal lens pressure may contribute to lens protein misfolding and/or unfolding, thereby leading to a loss of lens flexibility and the concomitant reduction in ability of the lens to focus.

It should be appreciated that, once any cellular protein is folded, various stress conditions may pose a threat to the protein's integrity. For instance, temperature variations, pressure, osmotic changes, antibiotics, solvents, and other chemicals and/or forces not only interfere with transcription, translation, and protein folding, but can also often disrupt the accurate three-dimensional protein structure.

It is generally known that pressure affects proteins, and more specifically, that pressure can unfold proteins. See, e.g., P. W. Bridgman, “The coagulation of albumin by pressure,” J Biol. Chem. 19:511-12 (1914); W. Kauzmann, “Thermodynamics of unfolding,” Nature 325:763-64 (1987).

Specifically, with respect to lens aggregation diseases, pressure may lead to precipitation and aggregation of proteins (e.g., lens proteins), cross-linking via disulfide bonds, reduction of glutathione, phosphorylation, and other post-translational protein modification factors that may cause progression of presbyopia and cataract.

Increased pressure may cause proteins to be penetrated by water in the same way they are penetrated by chemical agents (e.g., urea and guanidine) when such agents are added at high concentrations at atmospheric pressure. For instance, it is known that hyperbaric oxygen in vivo accelerates aging in the nuclear region of the guinea pig lens with regard to the loss of water-soluble and cytoskeletal proteins, damage to plasma membranes, formation of protein disulfide, and degradation of lens membrane protein MIP26. Such modifications are similar to those that occur in the nuclei of aging and cataractous human lenses.

In many cases, pressure drives proteins to unfold or misfold, resulting in intermediate, partially-folded protein conformations and molten-globular intermediates. Misfolded proteins, aggregates, and amyloids are usually derived from partially folded intermediates. Additionally, as compared to temperature, which affects a biological system's energy content, increased pressure generally shifts the equilibrium between associated and dissociated forms of proteins toward dissociation without affecting the system's energy content. Such increased pressure induces changes that range from small conformational effects, compressibility effects, and changes in populations of intermediate states to the complete loss of native folding of one or more proteins.

Additionally, in contrast to the low sensitivity of nucleic acids to pressure, lipid membranes are extremely sensitive to pressure, more so than water-soluble proteins. Alterations to lens membranes, including potentially as a result of oxidative processes, may contribute to development of lens aggregation diseases (e.g., cataracts). It is known that protein oxidation in lenses can initiate at lens membranes, and products of lipid oxidation in lenses can increase with both age and cataract development. It is further known that membrane derangement occurs in cataractous lenses in humans, potentially indicating that lipid oxidation and/or compositional changes in the membrane may cause lens opacification.

Various sources may cause such pressure inside the lens, leading to elevated internal lens pressure, as described in further detail below.

Hydrostatic Pressure

The space between the fiber cells of the lens is known as the interstitium. Fluid within the interstitium is termed the interstitial fluid. Such fluid circulates through the lens and fiber cells of the lens are accordingly bathed by, and within, the interstitial fluid. An intracellular gradient of hydrostatic pressure drives fluid from central fiber cells outward towards surface epithelial cells, pushing outwards against the lens capsule.

Mathematically, hydrostatic pressure is defined as a change in volume divided by a change in pressure. As a result, the more fluid that filters into the interstititum, the greater the volume of the interstitial space (Vᵢ) and the hydrostatic pressure within that space (PO. For instance, in young, healthy human lenses, an intracellular hydrostatic pressure gradient is from ˜340 mmHg in central fiber cells to ˜0 mmHg in surface cells.

Intralenticular (that is, located within the lens of the eye) hydrostatic pressure generally increases with age. Generally, as a consequence of accommodation, there is a tendency for water to move from the lens to the surrounding area(s), which then increase the osmolarity of the lens. The ratio of free water to bound water decreases with increasing pressure, and accordingly increases with decreasing pressure.

Accommodation often declines between the ages of forty and sixty. In the oldest normal human lenses, an increase in osmotic pressure causes the release of bound water to become free water. When such a response to pressure is irreversible, the released free water accumulates in so-called lakes.

This release of bound water from the hydration layers of macromolecules and its conversion to free water in condensed systems is known as syneresis, which is known as the extraction or expulsion of a liquid from a gel. In the lens, decreasing osmotic pressure induces syneresis.

During accommodation, liquid is expelled from its bound state with lens crystallins, thereby becoming free water, thus decreasing osmotic pressure. As the ability to accommodate is lost, the free water-to-bound water ratio decreases with increasing pressure, resulting in a significant syneretic response. The ability of the human lens to respond reversibly to pressure decreases with a decrease in accommodation. When the accommodation ability is lost altogether, an increase in free water, which may be a source of cataract formation, may ensue.

Younger lenses convert free water to bound water efficiently with increasing hydrostatic pressure, but, in older lenses, this ability is diminished and, in some cases, reversed. Generally, the total water content is much higher in cataractous lenses (that is, lenses affected by cataract) than normal lenses. Thus, in complete presbyopia (i.e., where there is no accommodation), the lens is fixed in its unaccommodated, compressed configuration, with a lower tendency for water movement out of the interstitium, thereby creating higher internal pressure. Therefore, with aging, the ability of the lens to compensate for increased hydrostatic pressure is decreased.

Compression

Another factor that may result in elevated internal lens pressure is compression that results via physical constraint of the lens.

Hydrostatic pressure affects other organs in the body besides the eye, including, for instance, the brain (which is encased by rigid bone) and the kidney (which is encased by a capsule). The lens, like the kidney, is confined by a capsule, and exists in a state in which small increases in fluid volume leads to large increases in pressure. Large increases in the interstitial pressure of tissue can lead to tissue damage and cellular death. Accordingly, constraint of the capsule surrounding the lens may add to increasing internal lens pressure.

In primates, the lens capsule itself is generally a strong, transparent membrane that is capable of shaping the lens and its surface curvature by participating in the process of accommodation. The capsule is an uninterrupted basement membrane completely enclosing the lens, sequestering the lens from other ocular tissues, protecting its optical integrity from penetration by large molecules and protecting the lens from infectious microbes (e.g., viruses, bacteria).

As the lens is avascular, the capsule must also allow for the passive exchange of metabolic substrates and waste in and out of the lens.

The elastic modulus of the capsule must therefore be sufficiently higher than that of the lens substance in order to allow the forces applied by the ciliary muscles to mold the lens shape. The adult human lens capsule has an elastic modulus of approximately two thousand times higher than the cellular lens cortex and nucleus that it surrounds.

During accommodation, the zonules, which insert into the lens capsule, apply stress that has both parallel (e.g., stretching) and perpendicular (e.g., compressive) components. These discrete stresses are transformed by the capsule into a uniform stress that is approximately perpendicular to the lens surface. The transition from the unaccommodated to the accommodated state would include a reduction of stresses perpendicular to the lens surface.

Under uniaxial load, capsular elastic moduli at 10% strain increase with age until about age thirty-five, from around 0.3 N/mm² to 2.3 N/mm², and then becomes relatively constant thereafter. In other words, past age thirty-five, the capsule load is maximized at around 2.3 N/mm².

On top of this, continual production of lens cell fibers by the lens epithelium in the environment constrained by the lens capsule, which is fixed in volume, accordingly contributes to continual crowding and compaction. The fluidic changes combined with continued pressure from the production of proteins in a confined space, result in increasing hydrostatic pressure with age and a syneretic process that continually increases resistance within the lens. This, in turn, leads to increased light scattering and a less pliable lens, decreasing the ability of the lens to accommodate, as seen in, e.g., presbyopia and other diseases.

Over time, there is an increase in lens stiffness and elastic modulus observed in the lens nucleus and cortex, resulting from the continual accession of fiber cells. As the elastic modulus of the lens substance increases, more force must be transmitted through the lens capsule to mold its shape. The inability of the lens capsule to achieve a sufficient elastic modulus over the lens substance in order to transmit the necessary forces for accommodation may be a key cause of presbyopia.

Effects of Pressure on Cellular Structure and Proteins

Pressure has also been shown to accelerate age-related loss of specific nuclear cytoskeletal proteins when compared to levels present in age-matched controls. These proteins include, for instance, actin, vimentin, ankyrin, alpha-actinin, and tubulin. Disulfide-crosslinking appears to be a primary cause of cytoskeletal protein loss. Additionally, pressure has been implicated as a cause of the disulfide cross-linking of MIP26 and other cytoskeletal proteins implicated in cataract development.

The effects of pressure may trigger the oxidative damage of gap junctions, which are clusters of intracellular channels that, when gated open, mediate the direct cell-to-cell transfer of low molecular mass (e.g., <1 kilodalton (kD)) substances including, for example, ions, secondary messengers, and nutritional metabolites. Since the lens lacks blood vessels, the lens must use non-vascular mechanisms to move nutrients into, and waste products out of, the fiber cells located in the center of the lens. This is achieved, in part, by an extensive network of gap junction channels that join the cells of the lens into what has been termed in the art as an ionic and metabolic syncytium. Indeed, mature fiber cells possess an exceptionally large number of gap junctions, likely the highest concentration in any tissue in the body.

The lens grows throughout life, so there is an age-dependent increase in lens size that impacts lens circulation. Increased size causes increases in intracellular gradients for, e.g., sodium, calcium, voltage, and hydrostatic pressure. Aging results in the accumulation of oxidative damage to membrane transport proteins, particularly fiber cell gap junction channels, which affect factors involved with lens circulation. Thus, aging has major effects on lens transport and homeostasis.

Gap Junction Coupling

The fibers in the center of the lens are uniquely dependent on intercellular communication with cells at the lens surface because they have no blood supply. Lens fibers have been shown to be joined into a syncytium with respect to ions. This syncytium is achieved by gap junctions between adjacent fibers, which permit ions and small transported metabolites to diffuse from cytoplasm to cytoplasm between adjacent cells.

Gap junctions themselves are composed of integral plasma membrane proteins known as connexins, three of which are present in the lenses of many species. The aforementioned age-related changes include the reduction in fiber cell gap junction coupling conductance, which can be caused by oxidative damage to lens connexins. Since fiber cell connexins are sensitive to oxidative damage and increases in lens size, the downregulation of gap junction coupling that occurs with age causes depolarization of the intracellular voltage and increases in intracellular concentrations of sodium and calcium. Depolarization and increased intracellular sodium concentration result in reductions of the transmembrane driving force of fiber cell membrane sodium-dependent transporters, which are also responsible for intracellular homeostasis. These effects compromise intracellular proteins and allow increased oxidative damage to crystallins and aggregation.

With age, the lens appears to compromise circulation in order to maintain a more constant pressure gradient, though such maintenance is imperfect. The age-dependent decline in the optical efficiency of the lens may be driven, at least in part, by changes in the lens circulation. Intracellular hydrostatic pressure in the lens is expected to vary in proportion according to the ratio of a²j_{Na}/Nⱼ, where a is the lens radius (in centimeters), j_{Na} is the average density of the fiber cell transmembrane influx of sodium (Na) (in moles per centimeter2), and Nⱼ is the number of open gap junction channels per area of fiber cell-to-cell contact (in centimeters⁻²).

Based on the above, and knowing that the number of open gap junction channels per area of fiber cell-to-cell contact decreases with age, it is possible to calculate that the hydrostatic pressure in the lens increases with age.

For example, it is known that, in lenses in which gap junction coupling is increased, the central pressure is lower. See, e.g., J. Gao et al., “The Effects of Age on Lens Transport,” Invest. Ophthalmol. Vis. Sci. 2013, Vol. 54, pages 7174-7187. However, if gap junction coupling is reduced, the central pressure is higher but the surface pressure is always zero. See, e.g., id. Thus, reduction in gap junction coupling is a direct cause of the age-related increase in intracellular hydrostatic pressure in the lens nucleus.

As coupling conductance decreases, forces that drive lens circulation must increase in order to maintain the circulating fluxes. For example, there must be increases in the intracellular hydrostatic pressure gradient, in the diffusion gradient for sodium, and in the voltage gradient. Both the accumulation of intracellular sodium and the depolarization of the intracellular voltage due to age leads to a reduction in the fiber cell transmembrane electrochemical potential for sodium entry. Consequently, sodium influx decreases and, since this drives water flow, water flow is also decreased, thereby moderating the increase in gradients for, e.g., intracellular sodium concentration, voltage, and hydrostatic pressure. Additionally, hydraulic conductivity depends on the number of open gap junction channels. Thus, both reduced hydraulic conductivity and increased radius of the lens cause the intracellular hydrostatic pressure (known as PO to increase with age.

Coupling is also essential for fiber cell homeostasis, since uncoupled mature fibers (MF) depolarize and subsequently become opaque. This increase in hydrostatic pressure, in addition to decreased coupling conduction, is an initiator of the oxidative processes described above herein that lead to lens degeneration diseases (e.g., presbyopia and cataract). Gap junctions in the MF survive for the lifetime of the organism without protein turnover.

It is known that α3 connexin has a role in coupling MF to peripheral cells. It is possible that α3 connexin provides long-term communication in mature fibers; thus, α3 connexin may help maintain lens transparency. For instance, lenses of so-called “knock-in” α3 mice, where α3 connexin is expressed under the endogenous α8 connexin locus, are transparent but smaller.

Cleavage of connexins occurs abruptly between the peripheral shell of differentiating fibers (DF) and the inner core of MF. The appearance of the cleaved connexins is generally correlated to a change in coupling conductance. The DF remains coupled, but conductance is reduced to 30 to 35% of normal. However, the gap junctions in the DF of α3 negative/negative lenses remained sensitive to pH. It is possible that there is active transport by peripheral cells to maintain a homeostatic environment for cells in the MF zone. Loss of coupling would cut off the MF zone from this circulation.

Role of Calcium

The reduction in gap-junction coupling mentioned above herein results in the increased retention of calcium, which causes an osmotic imbalance that leads to an inflow of water into spaces in-between the lens fibers. This can then contribute to the onset of presbyopia and/or cataract.

At the lens surface, calcium (Ca)-adenosine triphosphatase (Ca-ATPase) and sodium (Na)/Ca exchange occurs. This sets up circulation of Ca²⁺, where the path to the lens surface depends on the presence of gap junctions.

Multiple plasma membrane Ca²⁺-ATPase isoforms are expressed from four PMCA (plasma membrane calcium ATPase) genes (specifically, PMCA1-4) and alternative messenger RNA (mRNA) splicing. All four PMCA genes are expressed in the lens epithelium, the PMCA3 transcript being the most abundant. The transcripts for PMCA1, PMCA2, and PMCA4 also exist in decreasing order of abundance. PMCA assists in the extrusion of calcium from cells.

Gap junction coupling of interior fiber cells to the surface cells is an essential component of Ca homeostasis. The MF deep within the lens do not have Ca-ATPase activity or Na/Ca exchange to transport calcium out, yet have membranes that are permeable to calcium. Hence, calcium is continuously leaking into these cells throughout the volume of the lens. Ca²⁺ accumulates in MF until its diffusion to surface balances the equilibrium.

Loss of coupling cuts off the MF zone from this circulation. With time, ion gradients in uncoupled cells dissipate, intracellular calcium increases, proteolysis of cytoplasmic proteins (such as one or more crystallins) occurs, and denatured proteins aggregate and form light-scattering elements that may be responsible for nuclear opacity. Indeed, it is known in the art that elevated lens calcium and cataract are correlated, and calcium is also known to produce aggregation of proteins when added to solutions of soluble lens proteins.

Further, a progressive loss in cytoskeletal proteins is correlated with an increase in free calcium, causing most spectrin and vimentin (both of which are types of structural and/or cytoskeletal proteins) present in the lens cortex to disappear. This indicates that proteolysis by calcium-dependent enzymes (e.g., calpain) may play a significant role in cytoskeletal regulation and metabolism in the lens.

Calcium-induced transparency loss has at least two phases. At moderately increased calcium levels, opacification occurs without major degradation of various intracellular proteins and may be the result of calcium-stimulated interactions between the membrane-cytoskeletal network and one or more crystallins. Such a calcium-induced interaction between structural elements of the lens could be reversible.

Role of Cyclic Adenosine Monophosphate (cAMP)

Intracellular levels of cAMP are regulated by the balance between the activities of two enzymes, adenylyl cyclase (AC) and cyclic nucleotide phosphodiesterase (PDE). cAMP can bind to, and modulate the function of, a family of cyclic-nucleotide-gated ion channels. These are relatively non-selective cation channels that conduct calcium. Calcium stimulates calmodulin (CaM) and CaM-dependent kinases and, in turn, modulates cAMP production by regulating the activity of ACs and PDEs.

The CREM (cAMP responsive element modulator) gene, which encodes for the cAMP responsive element modulator protein, is also known to encode the powerful repressor also encodes the powerful repressor ICER (inducible cAMP early repressor), which negatively feeds back on cAMP-induced transcription.

Role of Ligands that Enhance Gap Junction Communication

Ligand-gated ion channels (referred to alternatively as “LIC” or “LGIC”), also known as ionotropic receptors, are transmembrane ion-channel proteins that open. By opening, the LICs allow ions (e.g., Na⁺, K⁺, Ca²⁺, and/or Cl⁻) to pass through the membrane in response to the binding of a chemical messenger (e.g., a ligand such as, for instance, a neurotransmitter). The LIC superfamily includes, for example, nicotinic acetylcholine receptors (nAChRs), adenosine triphosphate (ATP) receptors, γ-aminobutyric acid (GABA) receptors, glutamate receptors, glycine receptors, and 5-hydroxytryptamine (5-HT) receptors.

Various ligands that may enhance gap junction communication include, for instance, aquaporins, protein kinase C (PKC), filensin, pigment epithelium-derived factor (PEDF), and fibroblast growth factor (FGF), each of which will be described briefly below.

Lens epithelial cells express various aquaporins (e.g., aquaporin-1 (AQP1), aquaporin-5 (AQP5), and aquaporin-7 (AQP7)). AQP5 and AQP7 are expressed in lesser amounts than AQP1. Fiber cells also express, e.g., aquaporin-0 (AQP0) and AQP5, the latter of which is expressed in lesser amounts. AQP1 is the main aquaporin (AQP), representing a majority of AQPs in human lymphatic endothelial cells (HLEC). Further, AQP1 protein expression appears to be increased in HLEC from cataract patients. AQP5 protein expression may also similarly be increased. As a result, the regulation of AQP1 and/or AQP5 can help maintain lens transparency.

PKC positively regulates both water permeability and ionic conductance of AQP1 channels. Activation of PKC results in the phosphorylation of cellular proteins. Stimulation of PKC is dependent upon diacylglycerol (DAG) signal. PKCs are regulated by a variety of lipid secondary messengers, including diacylglycerol and phosphatidylserine. Further, phosphorylation of the enzyme plays a critical role in its activation.

The filensin and PEDF genes are known in the art to have important roles in the physiology and morphology of the transparent lens. Filensin is an intermediate filament protein and a component of the filament of lens fiber cells, while PEDF is a multifunctional secreted protein. Downregulation of the expression of the filensin and/or PEDF genes may therefore contribute to the formation of cataract.

FGF signaling is required for upregulation of gap junction mediated intercellular coupling (GJIC) at the lens equator. The mammalian FGF receptor (FGFR) family consists of four genes, specifically FGFR1 through FGFR4. Varieties of FGFR1 through FGFR4 proteins are produced that have differing affinities for ligands. Further, FGF has the ability to increase GJIC in cultured lens cells.

Further, gene modification may be used to regulate water, sodium, and/or calcium ion transport and storage of sarco-endoplasmic reticulum gating and lens fiber cell channels, thereby reducing or eliminating oxidative damage of gap junctions by upregulating gap junction coupling. Without wishing to be bound by theory, the aforementioned may occur via sustained activation of Extracellular Signal-Regulated Kinase (ERK).

Accordingly, without wishing to be bound by theory, an increase in internal lens hydrostatic pressure can result in several cascading effects, including, for instance, oxidation, connexin damage (e.g., to Cx43, Cx46, and/or Cx50), gap junction failure, a loss of lens circulation, calcium accumulation, and, finally, presbyopia and/or cataract.

Mitogen-activated Protein Kinase and Crystallin Phosphorylation

Phosphorylation processes appear to play a role in the onset and/or progression of various lens aggregation diseases, including, for instance, cataracts. Phosphorylation in the lens epithelium may be, at least on some levels and at initial stages, reversible.

Mitochondria in the lens cortex removes most oxygen, thereby maintaining low oxygen tension in the lens nucleus. During oxidative phosphorylation, some electron leakage to oxygen occurs, forming superoxide. Increased mitochondrial production of superoxide occurs with age. This process contributes to elevated H₂O₂ in the nucleus of older lenses.

Additionally, phosphorylation is the most common post-translational modification of various crystallins (e.g., the α-crystallins) in the human lens. Indeed, phosphorylation of αB-crystallin can result in uncontrolled protein aggregation.

It is known that crystallin αB, crystallin βA4, and phosphorylation and truncation of crystallins in the lens, all increase with the formation of lens opacity. Such increases may contribute to the formation of cataracts. Moreover, phosphorylation and truncation of these proteins increased with the progression of cataracts.

Some of the major protein modifications include, for example, phosphorylation of αB-crystallin at serine (Ser) residues 19, 45, and 59. In particular, the phosphorylation of αB-crystallin at Ser-45 results in uncontrolled aggregation.

Phosphorylation of α-crystallin subunits also occurs, and is likely catalyzed by a specific mitogen-activated protein kinases (MAP kinases or MAPKs), namely, MAP kinase-activated protein kinase (MAPKAP kinase)-2. MAP kinases, which regulate cellular properties in response to a wide range of extracellular stimuli, are known to phosphorylate the OH group of serine (Ser) or threonine (Thr) in proteins and play important roles in the regulation of cell proliferation, differentiation, survival, and apoptosis.

Additionally, there is increased localization of phosphorylated αB-crystallin to the cytoskeleton. Chronic perturbation of the intermediate filament network, and/or disorganization and/or disruption of other cytoskeletal networks, can result in the activation of p38 MAP kinase. This activation can lead to the aforementioned Ser-59 phosphorylation of αB-crystallin and its colocalization with cytoskeletal elements.

Ser-45 phosphorylation of αB-crystallin is mostly involved in the cell cycle, while Ser-59 phosphorylation of αB-crystallin prominently occurs under stress conditions. Thus, without wishing to be bound by theory, targeting one or more stress- or inflammation-related kinase pathways (e.g., the p38 MAP kinase pathway and the p44 kinase pathway) could result in the moderation of phosphorylation of αB-crystallin.

There is currently no known inhibitor for MAPKAP kinase-2 (which is a substrate of the p38 MAPK) that phosphorylates αB-crystallin. However, inhibition of either p38 MAPK or its activation (upstream to p38 MAPK) could be beneficial. Such inhibitors of p38 MAPK have been developed to target the kinase in the context of treating some human diseases (e.g., arthritis, cancers involving inflammation). Additionally, transient and/or temporary inhibition of the p38 MAP kinase pathway could also be beneficial, since low levels of phosphorylation of αB-crystallin may have protective effects. Such transient and/or temporary inhibition would also ameliorate the deleterious consequences, if any, of completely and/or irreversible inhibition of p38 MAPK.

Inhibition of Calpain and/or Calpain II

Calpains are Ca²⁺-dependent cysteine proteases. The unregulated Ca²⁺-mediated proteolysis of essential lens proteins by calpains might contribute to lens degeneration diseases (e.g., some forms of cataract) in both animals and humans. Further, the molecular structures of calpains have provided details that can be used to design calpain inhibitors. Such inhibitors have the potential to act as anti-cataract agents.

Five calpains are known in the art to occur in the lens, including calpain 1 (μ-calpain), calpain 2 (m-calpain), calpain 10, Lp82, and Lp85.

Additionally, the breakdown of vimentin in the outer cortex of the human lens, which occurs during aging, may be due to calpain. Bovine lens calpain 2 has been shown to degrade α-crystallin, actin, and vimentin, suggesting potential involvement of calpain 2 in the aging process. Both phenylmethylsulfonyl fluoride (PMSF) and leupeptin have also been shown to decrease vimentin degradation.

Further, proteolysis of lens cytoskeletal proteins (e.g., spectrin and vimentin) correlate with the loss of lens transparency. Lens proteins undergoing limited proteolysis by calpain may no longer interact properly, resulting in opacity.

Calpain inhibitors have been shown to reduce the rate of cataract formation. For instance, calpain 3 is necessary for the formation of age-dependent nuclear cataracts in α3Cx464^{-/-} mice. The loss of α3Cx46 leads to increased levels of Ca²⁺ ions, which activates Lp82/Lp85, thereby leading to the formation of a nuclear cataract.

Calpastatin, a calpain-specific inhibitor, is the endogenous inhibitor of at least two calpains, calpain 1 and calpain 2, the latter of which cleaves actin and vimentin. Additional exogenous calpain inhibitors are also being designed that focus on active-site-targeted small peptide analogues, which can provide a high selectivity for one or more calpains, coupled with good cell permeability and low toxicity. Without wishing to be bound by theory, at least some of these active-site-targeted small peptide calpain inhibitors act by binding the sulfhydryl group of cysteine (Cys) within the calpain active site, thereby inactivating the calpain.

Further, reducing calcium induced post-translational modification of proteins may be achieved by downregulating lens epithelial cell calcium influx or upregulating calcium efflux through controlling activation of calpastatin and/or the calpain family of enzymes. The aforementioned may be achieved by gene modification techniques, including, for instance, inhibiting the SRC kinase-dependent signaling pathway, inhibiting the calpain family of enzymes, and/or applying atropine.

Additionally, since human lens muscarinic receptors (e.g., in the iris, ciliary muscles, and retina) remain functional throughout life, they are also potential targets for cataract-inducing compounds.

Treatments for Reducing Lens Pressure

Accordingly, one or more embodiments of the present disclosure include compositions comprising one or more agents that reduce the presence and/or formation of proteins responsible for crowding, compacting, and/or causing increased internal lens pressure, as well as methods for using such compositions.

One or more of the compositions described herein may be used as, and/or integrated into, a topical and/or injectable treatment (e.g., eyedrops). Further, in one or more of the embodiments described herein, the composition may be administered via injection (e.g., retrobulbar injection, injection directly into the eye) in the form of a solution, suspension, emulsion, gel, prodrug, ointment, and/or sustained release vehicle (e.g., punctal plug, contact lens). Additionally, the composition may be administered directly into, and/or target, the lens of the eye, and more specifically the epithelium of the lens of the eye.

In at least one embodiment, an ophthalmologic composition is disclosed that reduces the effects of, retards, and/or eliminates one or more lens protein aggregation diseases (e.g., presbyopia, cataract). The composition may, in at least one example, regulate water, sodium, and/or calcium ion transport and/or storage through lens fiber cell channels. Without wishing to be bound by theory, the aforementioned results in minimizing and/or eliminating oxidative damage of gap junction channels and/or upregulating gap junction coupling. The composition may comprise one or more selective inhibitors of calcium ion (Ca²⁺) uptake into the endoplasmic reticulum (ER) (e.g., sarco/endoplasmic reticulum calcium-ATPase (SERCA) inhibitors, one or more Ca²⁺channel blockers (e.g., mibefradil, lanthanide ions (including, but not limited to, La³⁺, propranolol, felodipine, and verapamil), tetrandine, paxilline, artemisinin, prodrug mipsagargin antagonists, cyclopiazonic acid, 2,5-di(tert-butyl)hydroquinone (DBHQ, TBHQ), 1,3-dibromo-2,4,6-tris(methyl-isothio-uronium) benzene (Br2-TITU), cisplatin, curcumin, epiregulin, histidine decarboxylase, renin, DNA-damage-inducible transcript 4, oxidized low density lipoprotein (lectin-like) receptor 1, cofilin 1 (e.g., non-muscle), Hypoxia Inducible Factor 1 Subunit Alpha (HIF1A), forkhead box O3, ATP Binding Cassette Subfamily A Member 1 (ABCA1), Peptidyl Arginine Deiminase 3 (PADI3), Selenoprotein P, plasma, 1 (SEPP1), AKT Serine/Threonine Kinase 1 (AKT1), Glial Fibrillary Acidic Protein (GFAP), Nitric Oxide Synthase 3 (NOS3), Tyrosinase Related Protein 1 (TYRP1), insulin, Glycogen Synthase Kinase 3 Beta (GSK3B), Phospholipase A2 Group VI (PLA2G6), Cluster of Differentiation 69 (CD69), Staphylococcal Nuclease And Tudor Domain Containing 1 (SND1), PTEN-Induced Kinase 1 (PINK1), Solute Carrier Family 3 Member 2 (SLC3A2), VCP-Interacting Membrane Selenoprotein (VIMP), Fyn, transient receptor potential cation channel subfamily V member 1 (TrpV1), vanilloid receptor (e.g., VR1), Cytochrome C, Somatic (CYCS), ATPase Sarcoplasmic/Endoplasmic Reticulum Ca²⁺ Transporting 1 (ATP2A1), ATPase Sarcoplasmic/Endoplasmic Reticulum Ca²⁺ Transporting 2 (ATP2A2), ATPase Sarcoplasmic/Endoplasmic Reticulum Ca²⁺ Transporting 3 (ATP2A3), Activating Transcription Factor 4 (ATF4), Activating Transcription Factor 6 (ATF6), interleukin-6 (IL-6), interleukin 8 (IL-8), Cyclin D1 (CCND1), DNA Damage Inducible Transcript 3 (DDIT3), Endoplasmic Reticulum To Nucleus Signaling 1 (ERN1), Eukaryotic Translation Initiation Factor 2 Subunit Alpha (EIF2S1), Activating Transcription Factor 3 (ATF3), Mitogen-Activated Protein Kinase 8 (MAPK8), Fos Proto-Oncogene, AP-1 Transcription Factor Subunit (FOS), Eukaryotic Translation Initiation Factor 2 Alpha Kinase 3 (EIF2AK3), FRAS1 Related Extracellular Matrix 2 (FREM2), Calreticulin (CALR), Heat Shock Protein Family A (Hsp70) Member 5 (HSPA5), Caspase 3 (CASP3), Caspase 12 (CASP12), X-Box Binding Protein 1 (XBP)).

In at least one embodiment, an ophthalmologic composition comprises one or more non-selective cation channel blockers such as, for instance, verapamil derivates, flufenamic acid, Transient receptor potential (TRP) channel blockers (e.g., 2-aminoethoxydiphenyl borate (2-APB), N-(p-amylcinnamoyl)anthranilic acid (ACA), SKF 96365), Acid-Sensing Ion Channel (ASIC) blockers, Hyperpolarization-activated cyclic nucleotide-gated (HCN) channel blockers, and/or ZD7288.

In at least one embodiment, an ophthalmologic composition comprises one or more compounds that decrease activation and/or inhibit lens tyrosine kinases. Non-limiting examples of such compounds include tyrosine kinase (TK) inhibitors, specifically those that inhibit protein kinase A (PKA) and protein kinase C (PKC). Such inhibitors include, for instance, imatinib mesylate (also known by the trade name Gleevec®), Dasatinib (also known by the trade name Sprycel®), Nilotinib (also known by the trade name Tasigna®), Bosutinib (also known by the trade name Bosulif®), axitinib (also known by the trade name Inlyta®), erlotinib (also known by the trade name Tarceva®), pazopanib (also known by the trade name Votrient®), sunitinib (also known by the trade name Sutent®), gefitinib, genistein, lavendustin A, lavendustin C, PP1-AG1872, PP2-AG1879, SU6656, CGP77675, PD166285, cetuximab, UCS15A, p60-v-Src inhibitor peptide herbimycin A, and radicicol.

In at least one embodiment, an ophthalmologic composition comprises one or more agents that block calmodulin (CaM) binding to RyR2 (e.g., calmodulin antagonists). Such agents include, for instance, magnesium, CGS-19755, alpha-methyl-4-carboxyphenylglycine (MCPG), NB QX, ketamine, LY466195, tezampanel (NGX424), ADX10059, Perampanel, Dizocilpine (MK-801), Memantine (MEM), nitromemantines, Amantadine, cyanquixaline (6-cyano-7-nitroquinoxaline-2,3-dione) (CNQX), D-APV, NCT02136914, Talampanel, Orphenadrine, Meperidine, Agmatine, Ifenprodil, Tenocyclidine, Haloperidol, Dextromethorphan, and a combination of Dextromethorphan and Quinidine.

In at least one embodiment, an ophthalmologic composition comprises one or more agonists of CaM-dependent phosphodiesterase activity. Such agonists include, for instance, Theophylline, Papaverine, Alkylxanthines (e.g., Xanthine), Curcumin, Zardaverine, (R,S)-Rolipram, Filaminast, (R,S)-Mesopram, Cilomilast, Rofllumilast, Piclamilast, Sildenafil, Vardenafil, and Tadalafil.

In at least one embodiment, an ophthalmologic composition comprises one or more agents that inhibit expression of calmodulin. Such agents include, for instance, Phenothiazines, Trifluoperazine (e.g., 0.5-1% concentration), Thioridazine (e.g., 2% concentration), chlorpromazine, A-7 hydrochloride, calmidazolium chloride, CGS 9343B, KN-93, W-7 hydrochloride, W-5 hydrochloride, W-13 hydrochloride, and one or more calmodulin inhibitors (e.g., A1, F12, F14, G2, G3, H1, E6 Berbamine).

In at least one embodiment, an ophthalmologic composition comprises one or more agents that suppress Thapsigargin, including, for instance, ethylene glycol-bis(β-aminoethyl ether)-N,N,N′,N′-tetraacetic acid) (EGTA) and actinomycin.

In at least one embodiment, an ophthalmologic composition comprises one or more agents that enhance the activity of, and/or upregulate, adenylate cyclase (AC) activity. Such agents include, for instance, Colforsin Daropate Hydrochloride (NKH447). Without wishing to be bound by theory, enhancing the activity of, and/or upregulating, AC activity may affect upstream G-protein-coupled receptors (GPCR).

In at least one embodiment, an ophthalmologic composition comprises one or more agents that induce anti-calpain activity including, for instance, E64, AK295, SJA6017, and MDL 28170.

In at least one embodiment, an ophthalmologic composition comprises one or more

Transglutaminase (TGase) inhibitors including, for instance, Putrescine, Cystamine, Spermidine, Histamine, Monodansyl, Cadaverine, 5-(Biotinamido)pentylamine, Fluorescein, GppCp (GMPPCP), LDN-27219, Iodoacetamide, 3-halo-4,5-dihydroisoxazoles, α,β-unsaturated amides, epoxides, 1,2,4-thiadiazoles, maleimides, chloroacetamides, Cbz-gln-gly analogs (e.g., Cbz-gln(epoxide) and Cbz-gln(α,β-unsaturated)-gly), 6-Diazo-5-oxo-norleucine (DON), and 2-[(2-oxopropyl)thio]imidazolium.

In at least one embodiment, an ophthalmologic composition comprises one or more antioxidants and/or inhibitors of reactive oxygen species-generating enzymes (e.g., nitric oxide synthetase inhibitors) including, for instance, aminoguanidine and disulfiram.

In at least one embodiment, an ophthalmologic composition comprises one or more inhibitors of

Myosin Light Chain (MLC) Kinase including, for instance, Peptide 18, ML-7, ML-9, K-252a, KT592, Wortmannin, Quercetin, Genistin, Wogonin, Capsaicin, Salvianolic acid B, Lithium, MLCK (342-352) amide.

In at least one embodiment, an ophthalmologic composition comprises one or more Inositol 1,4,5-Trisphosphate (InsP₃) receptor inhibitors including, for instance, Xestospongin and 2-Aminoethoxydiphenyl borate (2-APB).

In at least one embodiment, an ophthalmologic composition comprises one or more Inositol 1,4,5-trisphosphate 3-kinase (IP3K) inhibitors and/or one or more anti-IP3K-specific antibodies. The aforementioned include, for instance, adriamycin 63.

In at least one embodiment, an ophthalmologic composition comprises one or more agents that reduce gap junction uncoupling and/or increase gap junction coupling. Such agents include, for instance, D-Limonene, Fisetin, Honokiol, Epigallocatechin gallate, Grape Seed Proanthocyanidin, and Decorin. Without wishing to be bound by theory, these agents may reduce calcium-induced post-translational modifications of proteins by downregulating lens epithelial cell calcium influx and/or upregulating calcium efflux through sustained activation of Extracellular Signal-Regulated Kinase (ERK) proteins.

In at least one embodiment, an ophthalmologic composition comprises one or more agents that inhibit and/or downregulate protein kinase Cγ (pkCγ) including, for instance, one or more proteins from the growth factor receptor bound (Grb)2-associated binder (Gab)/Daughter of Sevenless (DOS) family, one or more of the 14-3-3 proteins, one or more proteins from the insulin receptor substrate (IRS) family (e.g., IRS-1-6), one or more proteins from the fibroblast growth factor (FGF) receptor substrate 2 (FRS2) family, and one or more proteins from the downstream of tyrosine kinases (Dok) families.

In at least one embodiment, an ophthalmologic composition comprises one or more ligands that activate G-protein signaling molecules. Such ligands include, for instance, Secretin, Glucagon and Glucagon-like peptides (e.g., GLP-1 and GLP-2), Growth Hormone-Releasing Hormone (GHRH), Pituitary Adenylate Cyclase activating peptide (PACAP), Corticotropin-Releasing Hormone (CRH), Vasoactive Intestinal Peptide (VIP), Parathyroid Hormone (PTH), Tuberoinfundibular Peptide of 39 Residues (TIP-39), Adrenomedullin, Calcitonin-Related Peptides, Calcitonin, Amylin (AMY), Calcitonin gene-related peptide (CGRP), and Receptor activity-modifying proteins (e.g., RAMP1, RAMP2, RAMP3).

In at least one embodiment, an ophthalmologic composition comprises one or more agents that activate Protein Kinase A (PKA) (e.g., cAMP agonists, PKA activators). Such agents include, for instance, Sp-cAMP, dbcAMP, 15(R)-prostaglandin D2, 6-Bnz-cAMP, Colforsin Daropate Hydrochloride (NKH447), PDE4D, AN2728, Roflumilast, Forskolin (FSK) derivatives, cilomilast, AN2728, rolipram, apremilast, the Adenylyl Cyclase (ADCY) inhibitor MDL-12,330A, and one or more 2-arylpyrimidine derivatives.

In at least one embodiment, an ophthalmologic composition comprises one or more agents that inhibit and/or downregulate cAMP-dependent PDE. Such agents include, for instance, milrinone, inamrinone (formerly known as amrinone), cilostazol, sildenafil, and tadalafil.

In at least one embodiment, an ophthalmologic composition comprises one or more agents that activate, stimulate, and/or upregulate Calpastatin. Such agents include, for instance, Cimaterol, Clenbuterol, and other beta-agonists.

In at least one embodiment, an ophthalmologic composition comprises one or more agents that control the activity of Calcium-dependent proteases (e.g., m-calpain, Lp82). Such agents include one or more Cysteine protease inhibitors such as, for example, E-64, AK295, SJA6017, and MDL 28170.

In at least one embodiment, an ophthalmologic composition comprises one or more agents that inhibit the action of one or more proteases. Such agents include one or more protease inhibitors such as, for example, phenylmethylsulfonyl fluoride (PMSF), Leupeptin, Pepstatin, Ethylene glycol tetraacetic acid (EGTA), and lodoacetate.

In at least one embodiment, an ophthalmologic composition comprises one or more agents that decrease phosphorylation of α,β-crystallin and/or human lymphatic endothelial cells (HLECs) such as, for example, one or more inhibitors of various tyrosine kinases (e.g., by applying inhibitors of the tyrosine kinases including MLC Kinase, SRC Kinase, and MAP Kinase (MAPK) p38 kinase). The one or more agents may be, for instance, MAPK p38 kinase inhibitors including, for example, MicroRNA-182-5p, Decorin, D-Limonene, Fisetin, Honokiol, Epigallocatechin gallate, Grape seed proanthocyanidin, Dilmapimod, SRC Kinase inhibitor PP1, p38α, AMG-548, SC10-469, SC10-323, VX-702, Adezmapimod (SB203580), Doramapimod (BIRB 796), SB202190 (FHPI), Ralimetinib (LY2228820) dimesylate, PH-797804, Neflamapimod (VX-745), TAK-715, 3′-Hydroxypterostilbene, Pamapimod, SD0006, SB239063, Skepinone-L, Losmapimod (GW856553X), AUDA (compound 43), R1487, Trans-Zeatin, Praeruptorin A, SEA0400, Mulberroside A, BMS-582949, TA-01, UM-164, PD 169316, Metformin hydrochloride (Metformin HCl), Asiatic Acid (Dammarolic acid), Berberine chloride (NSC 646666), ML141, Rotundic acid, 5′-N-Ethylcarboxamidoadenosine (NECA), Pexmetinib (ARRY-614), trans-1-(4-hydroxycyclohexyl)-4-(4-fluorophenyl)-5-(2-methoxypyridimidin-4-yl)imidazole (SB 239063), FR-167653, SB-681323, Angiotensin 1, Angiotensin 2, Angiotensin 3, Angiotensin 4, Angiotensin 5, Angiotensin 6, Angiotensin 7, PHA666859, AZD7624, SD-282 (ICS-p38IH), ML3403, Vitamin E analog 7, LN950, CBS3830, JLU1124, Rituximab, RO4399247, AVE8677, Compound 37, Peptide 11R-p381110, Prexasertib, and U0126.

In at least one embodiment, an ophthalmologic composition comprises one or more agents that disable ER signaling including, for instance, mitogen-activated protein kinase enzymes MEK1 and/or MEK2, and one or more MEK inhibitors (e.g., U0126). Without wishing to be bound by theory, such agents may stop cell division, limit lens fiber cell growth, and interrupt the MAPK pathway.

In at least one embodiment, an ophthalmologic composition comprises one or more agents that reduce or eliminate the breakdown of Acetylcholine in the lens of the eye.

In at least one embodiment, an ophthalmologic composition comprises one or more Cholinesterase inhibitors including, for instance, Aricept (Pro), rivastigmine, donepezil, donepezil and memantine, donepezil, and galantamine.

In at least one embodiment, an ophthalmologic composition comprises one or more muscarinic agonists such as, for example, Atropine, pilocarpine, aceclidine, carbachol, scopolamine, glycopyrrolate, Bethanechol, Cevimeline, NGX267, Methacholine, Xanomeline, and ipratropium bromide. It should be appreciated that, although one or more of the aforementioned agonists are generally known in the art, none have been used to inhibit lens protein aggregation diseases. For example, Atropine is only generally known in the art for myopia control.

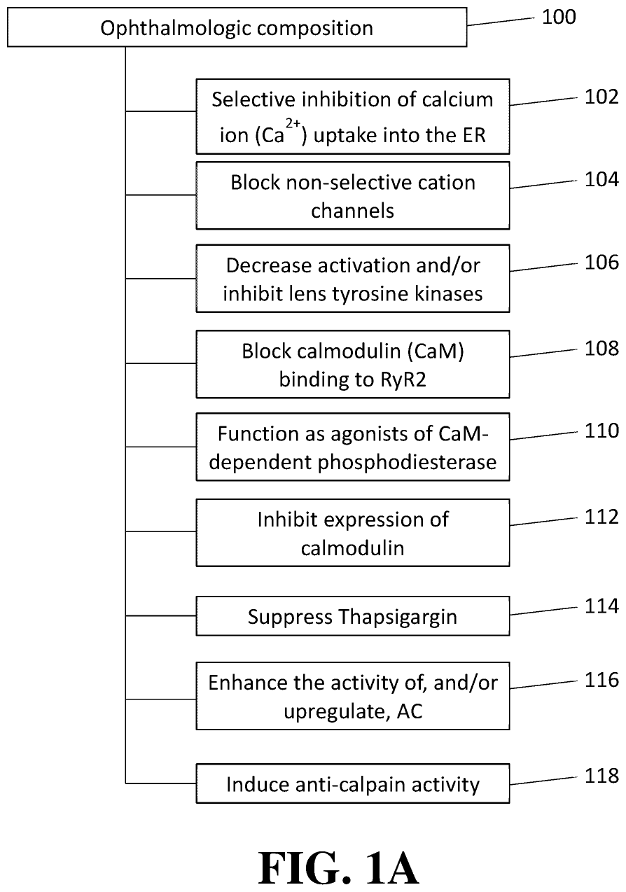

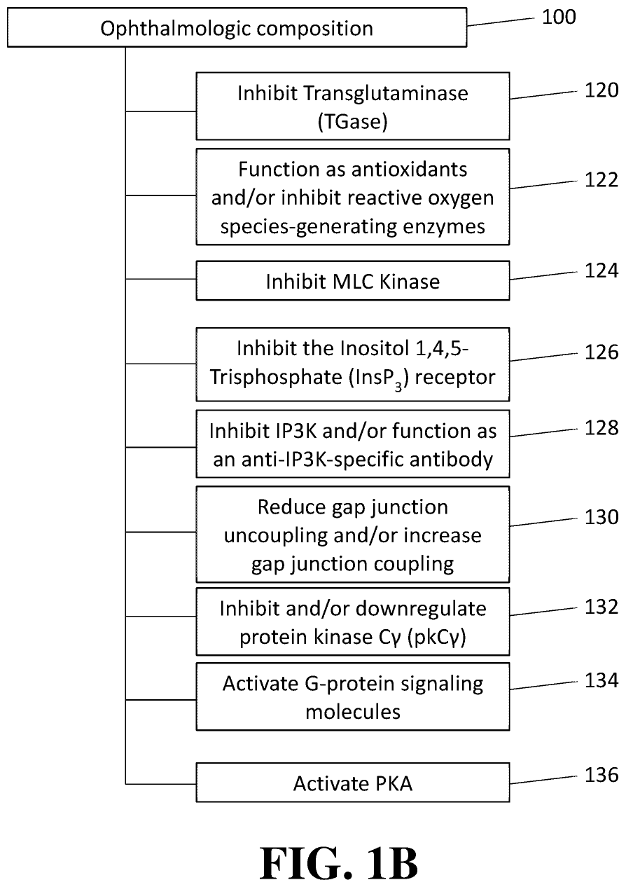

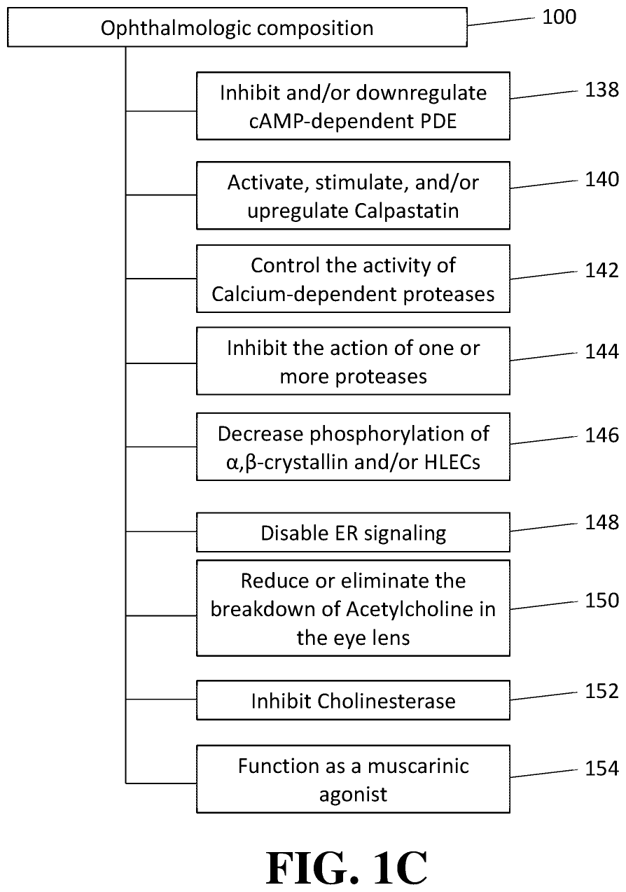

Therefore, turning now to FIGS. 1A-1C, an ophthalmologic composition 100 is shown that may comprise one or more of agents, compounds, and/or molecules with the functions shown at blocks 102, 104, 106, 108, 110, 112, 114, 116, 118, 120, 122, 124, 126, 128, 130, 132, 134, 136, 138, 140, 142, 144, 146, 148, 150, 152, and 154.

Specifically, FIG. 1A shows that the ophthalmologic composition 100 that may comprise one or more agents, compounds, and/or molecules that have one or more of the following functions: one, selective inhibition of calcium ion (Ca²⁺) uptake into the endoplasmic reticulum (ER) (shown at block 102), two, block non-selective cation channels (shown at block 104), three, decrease activation and/or inhibit lens tyrosine kinases (shown at block 106), four, block calmodulin (CaM) binding to RyR2 (e.g., calmodulin antagonists) (shown at block 108), five, function as agonists of CaM-dependent phosphodiesterase activity (shown at block 110), six, inhibit expression of calmodulin (shown at block 112), seven, suppress Thapsigargin (shown at block 114), eight, enhance the activity of, and/or upregulate, adenylate cyclase (AC) activity (shown at block 116), and nine, induce anti-calpain activity (shown at block 118).

FIG. 1B shows that the ophthalmologic composition 100 that may comprise one or more agents, compounds, and/or molecules that have one or more of the following functions: ten, inhibit Transglutaminase (TGase) (shown at block 120), eleven, function as antioxidants and/or inhibit reactive oxygen species-generating enzymes (shown at block 122), twelve, inhibit MLC Kinase (shown at block 124), thirteen, inhibit the Inositol 1,4,5-Trisphosphate (InsP₃) receptor (shown at block 126), fourteen, inhibit Inositol 1,4,5-trisphosphate 3-kinase (IP3K) and/or function as an anti-IP3K-specific antibody (shown at block 128), fifteen, reduce gap junction uncoupling and/or increase gap junction coupling (shown at block 130), sixteen, inhibit and/or downregulate protein kinase Cγ (pkCγ) (shown at block 132), seventeen, activate G-protein signaling molecules (shown at block 134), and eighteen, activate PKA (shown at block 136).

FIG. 1C shows that the ophthalmologic composition 100 that may comprise one or more agents, compounds, and/or molecules that have one or more of the following functions: nineteen, inhibit and/or downregulate cAMP-dependent PDE (shown at block 138), twenty, activate, stimulate, and/or upregulate Calpastatin (shown at block 140), twenty-one, control the activity of Calcium-dependent proteases (shown at block 142), twenty-two, inhibit the action of one or more proteases (shown at block 144), twenty-three, decrease phosphorylation of α,β-crystallin and/or human lymphatic endothelial cells (HLECs) (shown at block 146), twenty-four, disable ER signaling (shown at block 148), twenty-five, reduce or eliminate the breakdown of Acetylcholine in the lens of the eye (shown at block 150), twenty-six, inhibit Cholinesterase (shown at block 152), and twenty-seven, function as a muscarinic agonist (shown at block 154).

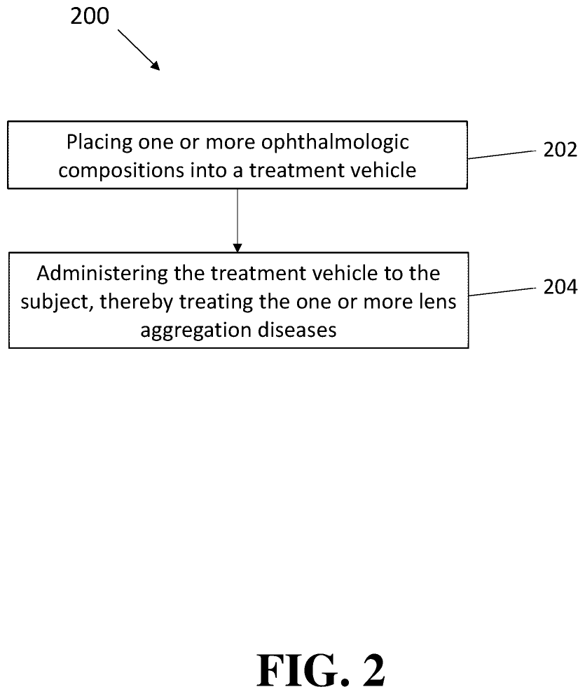

Turning now to FIG. 2, a method 200 is shown for treating a subject having one or more lens aggregation diseases, the method comprising, at block 202, placing one or more ophthalmologic compositions, which may be any of the ophthalmologic compositions described herein, into a treatment vehicle; and, at block 204, administering the treatment vehicle to the subject, thereby treating the one or more lens aggregation diseases. The treatment vehicle may be a solution, suspension, emulsion, gel, prodrug, ointment, and/or sustained release vehicle (e.g., punctal plug, contact lens). Further, the administering may be performed directly into the subject's eye, including the eye lens and/or the epithelium of the eye lens.

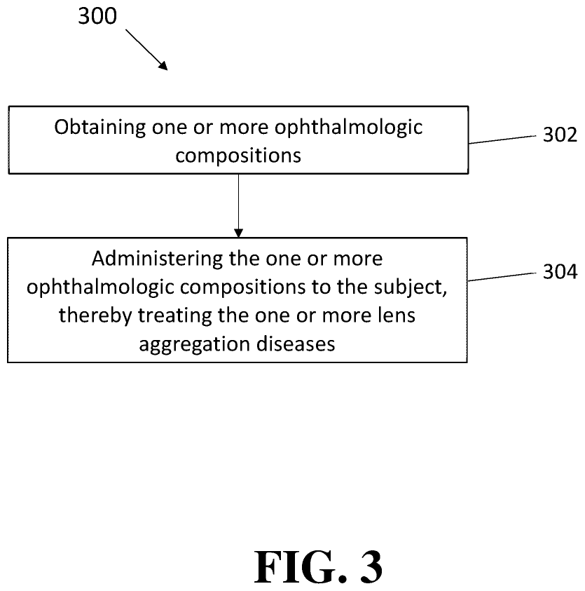

Turning now to FIG. 3, a method 300 is shown for treating a subject having one or more lens aggregation diseases, the method comprising, at block 302, obtaining one or more ophthalmologic compositions; and, at block 304, administering the one or more ophthalmologic compositions to the subject, thereby treating the one or more lens aggregation diseases. The obtaining step may comprise, for instance, creating or generating the one or more ophthalmologic compositions. These one or more ophthalmologic compositions may be any of the ophthalmologic compositions described herein. Additionally, the one or more ophthalmologic compositions may be provided in a solution, suspension, emulsion, gel, prodrug, ointment, and/or sustained release vehicle (e.g., punctal plug, contact lens). Further, the administering may be performed directly into the subject's eye, including the eye lens and/or the epithelium of the eye lens.

These and other objectives and features of the invention are apparent in the disclosure, which includes the above and ongoing written specification.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated.

The invention is not limited to the particular embodiments illustrated in the drawings and described above in detail. Those skilled in the art will recognize that other arrangements could be devised. The invention encompasses every possible combination of the various features of each embodiment disclosed. One or more of the elements described herein with respect to various embodiments can be implemented in a more separated or integrated manner than explicitly described, or even removed or rendered as inoperable in certain cases, as is useful in accordance with a particular application. While the invention has been described with reference to specific illustrative embodiments, modifications and variations of the invention may be constructed without departing from the spirit and scope of the invention as set forth in the following claims.

1. A method for treating one or more lens aggregation diseases, the method comprising:
administering an ophthalmologic composition into a patient's eye lens epithelium, thereby treating one or more lens aggregation diseases,
wherein the ophthalmologic composition comprises at least one agent that at least one of:
regulates at least one of water, sodium, calcium ion transport, and calcium ion storage through lens fiber cell channels, and
reduces at least one of the presence and formation of proteins responsible for at least one of crowding, compacting, and causing increased internal lens pressure,
wherein the ophthalmologic composition has a form selected from the group consisting of: a solution, a suspension, or combinations thereof, and
wherein the one or more lens aggregation diseases comprises presbyopia.

2. The method of claim 1, wherein the at least one agent comprises at least one selective inhibitor of calcium ion (Ca²⁺) uptake into the endoplasmic reticulum (ER).

3. The method of claim 2, wherein the at least one selective inhibitor comprises at least one of a sarco/endoplasmic reticulum calcium-adenosine triphosphate (ATP)ase (SERCA) inhibitor and a Ca²⁺ channel blocker.

4. The method of claim 3, wherein the Ca²⁺ channel blocker is.

5. The method of claim 1, wherein the at least one agent comprises at least one non-selective cation channel blocker selected from the group consisting of: a verapamil derivate, flufenamic acid, 2-aminoethoxydiphenyl borate (2-APB), N-(p-amylcinnamoyl) anthranilic acid (ACA), SKF 96365, an Acid-Sensing Ion Channel (ASIC) blocker, a Hyperpolarization-activated cyclic nucleotide-gated (HCN) channel blocker, ZD7288, and combinations thereof.

6. The method of claim 1, wherein the at least one agent at least one of decreases activation and inhibits lens tyrosine kinases.

7. The method of claim 6, wherein the at least one agent comprises at least one tyrosine kinase inhibitors selected from the group consisting of: imatinib mesylate, dasatinib, nilotinib, bosutinib, axitinib, erlotinib, pazopanib, sunitinib, gefitinib, genistein, lavendustin A, lavendustin C, PP1-AG1872, PP2-AG1879, SU6656, CGP77675, PD166285, cetuximab, UCS15A, p60-v-Src inhibitor peptide herbimycin A, radicicol, genistein, and combinations thereof.

8. The method of claim 1, wherein the at least one agent blocks calmodulin (CaM) binding to RyR2.

9. The method of claim 8, wherein the at least one agent comprises at least one calmodulin antagonists selected from the group consisting of: magnesium, CGS-19755, alpha-methyl-4-carboxyphenylglycine (MCPG), NBQX, ketamine, LY466195, tezampanel (NGX424), ADX10059, Perampanel, Dizocilpine (MK-801), Memantine (MEM), nitromemantines, Amantadine, cyanquixaline (6-cyano-7-nitroquinoxaline-2,3-dione) (CNQX), D-APV, NCT02136914, Talampanel, Orphenadrine, Meperidine, Agmatine, Ifenprodil, Tenocyclidine, Haloperidol, Dextromethorphan, a combination of Dextromethorphan and Quinidine, and combinations thereof.

10. The method of claim 1, wherein the at least one agent comprises at least one agonist of CaM-dependent phosphodiesterase activity.

11. The method of claim 10, wherein the at least one agonist is selected from the group consisting of: Theophylline, Papaverine, an Alkylxanthine, Curcumin, Zardaverine, (R,S)-Rolipram, Filaminast, (R,S)-Mesopram, Cilomilast, Rofllumilast, Piclamilast, Sildenafil, Vardenafil, and Tadalafil.

12. The method of claim 1, wherein the at least one agent inhibits expression of calmodulin.

13. The method of claim 12, wherein the at least one agent is selected from the group consisting of: a Phenothiazine, a Trifluoperazine, a Thioridazine, chlorpromazine, A-7 hydrochloride, calmidazolium chloride, CGS 9343B, KN-93, W-7 hydrochloride, W-5 hydrochloride, W-13 hydrochloride, at least one calmodulin inhibitor, and combinations thereof.

14. A method for treating one or more lens aggregation diseases, the method comprising:
administering an ophthalmologic composition into a patient's eye lens epithelium, thereby treating one or more lens aggregation diseases,
wherein the ophthalmologic composition comprises at least one agent that at least one of:
regulates at least one of water, sodium, calcium ion transport, and calcium ion storage through lens fiber cell channels, and
reduces at least one of the presence and formation of proteins responsible for at least one of crowding, compacting, and causing increased internal lens pressure, and
wherein the one or more lens aggregation diseases comprises presbyopia.

15. The method of claim 14, wherein the at least one agent suppresses Thapsigargin.

16. The method of claim 15, wherein the at least one agent is selected from the group consisting of: ethylene glycol-bis(β-aminoethyl ether)-N,N,N′,N′-tetraacetic acid) (EGTA), actinomycin, and combinations thereof.

17. The method of claim 14, wherein the at least one agent at least one of enhances the activity of, and upregulates, adenylate cyclase (AC) activity.

18. The method of claim 17, wherein the at least one agent comprises Colforsin Daropate Hydrochloride (NKH447).

19. The method of claim 14, wherein the at least one agent induces anti-calpain activity.

20. The method of claim 19, wherein the at least one agent is selected from the group consisting of: E64, AK295, SJA6017, MDL 28170, and combinations thereof.

21. The method of claim 14, wherein the at least one agent inhibits Transglutaminase (TGase) activity.

22. The method of claim 21, wherein the at least one agent is selected from the group consisting of: Putrescine, Cystamine, Spermidine, Histamine, Monodansyl, Cadaverine, 5-(Biotinamido)pentylamine, Fluorescein, GppCp (GMPPCP), LDN-27219, Iodoacetamide, a 3-halo-4,5-dihydroisoxazole, an α,β-unsaturated amide, an epoxide, a 1,2,4-thiadiazole, a maleimide, a chloroacetamide, a Cbz-gln-gly analog, 6-Diazo-5-oxo-norleucine (DON), 2-[(2-oxopropyl)thio]imidazolium, and combinations thereof.

23. The method of claim 14, wherein the at least one agent comprises at least one of at least one antioxidant and inhibitors of reactive oxygen species-generating enzymes.

24. The method of claim 23, wherein the at least one agent is selected from the group consisting of: aminoguanidine, disulfiram, and combinations thereof.

25. The method of claim 14, wherein the at least one agent inhibits Myosin Light Chain (MLC) Kinase.

26. The method of claim 25, wherein the at least one agent is selected from the group consisting of: Peptide 18, ML-7, ML-9, K-252a, KT592, Wortmannin, Quercetin, Genistin, Wogonin, Capsaicin, Salvianolic acid B, Lithium, MLCK (342-352) amide, and combinations thereof.
